(19) United States
(12) Patent Application Publication
(10) Pub. No.: US 2025/0156606 A1
(43) Pub. Date: May 15, 2025

(54) EVALUATING CARBON SEQUESTRATION CONTRIBUTION OF NATURE-BASED ASSETS LOCATED IN WATER ENVIRONMENTS

(71) Applicant: INTERNATIONAL BUSINESS MACHINES CORPORATION, ARMONK, NY (US)

(72) Inventors: Fearghal O'Donncha, Aran Islands (IE); Adam James Thompson, Ruderting (DE); Moritz Johannes Stabler, Koln (DE); Andrew James Stanford-Clark, Chale (GB); Ricardo Martinho, Lisbon (PT); Jonathan Wright, New York, NY (US)

(21) Appl. No.: 18/543,364

(22) Filed: Dec. 18, 2023

(30) Foreign Application Priority Data
Nov. 15, 2023 (GB) 2317512.8

(51) Int. Cl. G06F 30/27

(52) U.S. Cl. CPC G06F 30/27

(57) ABSTRACT

A method, computer program product, and computer system are provided for evaluating carbon sequestration contributions of nature-based assets located in water environments. The method includes: obtaining meta-descriptors of a nature-based asset and a region of a water environment in which the asset is located; mapping dimensions of the nature-based asset; accessing data of monitored physical characteristics of the nature-based asset in the water environment based on the meta-descriptors of the asset; accessing data of monitored carbon concentration in the region of the water environment based on the meta-descriptors of the region; accessing data on monitored physical, chemical, and biological properties of the water environment based on the meta-descriptors of the region; and applying a model to the accessed data to assign a carbon sequestration contribution per unit measurement of the nature-based asset.

### BACKGROUND

The present invention relates to carbon sequestration, and more specifically, to evaluating carbon sequestration contribution of nature-based assets located in water environments.

The world's oceans are the largest sustained sink of carbon dioxide released to the atmosphere. It is now estimated that about 1.8 Pg C year⁻¹ go into the ocean, but the uncertainty is still quite large (˜0.7 Pg C year⁻¹).

Different regions of the oceans are not equivalent with respect to CO₂ absorption. Some regions, such as the Equatorial Pacific, release CO₂ to the atmosphere, whereas others like the North Atlantic absorb it. In addition to this regional variability, a large temporal variability can occur.

### SUMMARY

According to an aspect of the present invention there is provided a computer-implemented method for evaluating carbon sequestration contributions of nature-based assets located in water environments, said method comprising: obtaining meta-descriptors of a nature-based asset and a region of a water environment in which the asset is located; mapping dimensions of the nature-based asset; accessing data of monitored physical characteristics of the nature-based asset in the water environment based on the meta-descriptors of the asset; accessing data of monitored carbon concentration in the region of the water environment based on the meta-descriptors of the region; accessing data on monitored physical, chemical, and biological properties of the water environment based on the meta-descriptors of the region; and applying a model to the accessed data to assign a carbon sequestration contribution per unit measurement of the nature-based asset.

According to another aspect of the present invention there is provided system for evaluating carbon sequestration contributions of nature-based assets located in water environments, comprising: a processor and a memory configured to provide computer program instructions to the processor to execute the function of the components: a meta-descriptor component for obtaining meta-descriptors of a nature-based asset and a region of a water environment in which the asset is located; a mapping component for mapping dimensions of the nature-based asset; an asset data monitoring component for accessing data of monitored physical characteristics of the nature-based asset in the water environment based on the meta-descriptors; a carbon concentration data component for accessing data of monitored carbon concentration in the region of the water environment based on the meta-descriptors; a water environment data component for accessing data on monitored physical, chemical, and biological properties of the water environment based on the meta-descriptors of the region; and a model applying component for applying a model to assign carbon sequestration contribution per unit measurement of the nature-based asset.

According to a further aspect of the present invention there is provided computer program product for evaluating carbon sequestration contributions of nature-based assets in water environments, the computer program product comprising a computer readable storage medium having program instructions embodied therewith, the program instructions executable by a processor to cause the processor to: obtain meta-descriptors of a nature-based asset and a region of a water environment in which the asset is located; map dimensions of the nature-based asset; access data of monitored physical characteristics of the nature-based asset in the water environment based on the meta-descriptors of the asset; access data of monitored carbon concentration in the region of the water environment based on the meta-descriptors of the region; access data on monitored physical, chemical, and biological properties of the water environment based on the meta-descriptors of the region; and apply a model to the accessed data to assign a carbon sequestration contribution per unit measurement of the nature-based asset The computer readable storage medium may be a non-transitory computer readable storage medium and the computer readable program code may be executable by a processing circuit.

### BRIEF DESCRIPTION OF THE DRAWINGS

Embodiments of the present invention will now be described, by way of example only, with reference to the accompanying drawings:

FIG. 1 is a flow diagram of an example embodiment of a method in accordance with embodiments of the present invention;

FIG. 2 is a block diagram of an example embodiment of a system in accordance with embodiments of the present invention;

FIG. 3 is a flow diagram of an example embodiment of a method of training in accordance with embodiments of the present invention; and

FIG. 4 is a block diagram of an example embodiment of a computing environment for the execution of at least some of the computer code involved in performing the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

Embodiments of a method, system, and computer program product are provided for evaluating carbon sequestration contributions and other contributions of nature-based assets located in water. Nature-based assets located in water are natural or man-made resources that produces a positive environmental value. Examples include: natural or manmade coral reefs, mangrove forests, seagrass farm, oyster habitat, etc. The nature-based assets may also be referred to as nature-based solutions as they may be developed to solve the problem of carbon emissions.

The disclosed method and system use a model to assign a carbon sequestration contribution per unit measurement (e.g. per square unit of area or per cube unit of volume) of nature-based assets located in water based on inputs to the model. The model may be a trained artificial intelligence modeling system (e.g., transformer model network, graph neural network, seq2seq network, etc.). In other embodiments, the modeling system may be a rule-based model.

The disclosed method has the advantage of modeling to verify carbon sequestration contributions of nature-based assets using data gathered from an asset and its water environment. This provides accurate metrics on carbon absorption and storage critical for net-zero goals. The method uses direct measurement and not proxies to provide a robust, defensible, quantification metric.

The disclosed method and system: access data of monitored carbon concentration in a region of a water environment (for example, an ocean or other water body) by leveraging a sensing platform that may include artificial intelligence sensing devices; access data of monitored physical characteristics of a nature-based asset in the water environment; access data on monitored physical, chemical, and biological properties of the water environment; and map dimensions of the nature-based asset. This data is input into the modeling system to assign a carbon sequestration contribution per unit measurement of the nature-based asset.

The output is an evaluation of a nature-based asset. This may include metrics on: carbon sequestration, biodiversity, nature-based contribution of asset, physical extents and characteristics of nature-based asset, and nature-based asset health or status indices.

The method and system may use an immutable data store (e.g. blockchain) to store nature-based assets and associated time-varying carbon sequestration and/or other nature-based contribution in tokens. Such storage can infuse trust and transparency within the system.

The described method and system may be applied to combine observations, machine learning, and an immutable data store to validate and verify water-located (i.e. ocean- or freshwater-located) nature-based solutions, and tokenize nature-based contributions within a voluntary or compliance marketplace. This may provide a framework to distinguish baseline carbon sequestration from those that are a contribution of a given nature-based asset located in water, such as coral reef (natural or man-made) or mangrove forests. The method and system may simultaneously monitor the status or health of the nature-based asset.

The evaluating carbon sequestration contribution of nature-based assets located in water is an improvement in the technical field of environmental monitoring generally and more particularly in the technical field of environmental monitoring and evaluation for carbon sequestration purposes.

The following terminology is used in this description.

“Carbon sequestration” is the process of capturing, securing, and storing carbon dioxide from the atmosphere.

“Voluntary carbon markets” allow carbon emitters to offset their emissions by purchasing carbon credits emitted by projects targeted at removing greenhouse gas from the atmosphere or reducing their emissions.

“Compliance carbon markets” are created as a result of any national, regional and/or international policy or regulatory requirement.

“Nature-based solutions” are defined by the European Commission as “Solutions that are inspired and supported by nature, which are cost-effective, simultaneously provide environmental, social and economic benefits and help build resilience. Such solutions bring more, and more diverse, nature and natural features and processes into cities, landscapes and seascapes, through locally adapted, resource-efficient and systemic interventions”.

Carbon markets are systems in which countries, companies, or individuals can buy and sell carbon emission allowances or credits to meet their emission reduction targets. The primary stakeholders include governments, which set regulations and caps on emissions; companies that either emit greenhouse gases (and therefore need to buy credits) or reduce emissions (and can sell credits); and intermediaries or brokers facilitating the trading process. The market operates on the principle of supply and demand: entities needing to offset their emissions buy credits from those who have excess reductions, making money for sellers who can provide verifiable emissions reductions at costs lower than the prevailing market prices.

Estimates suggest that nature-based solutions can provide 37% of the mitigation needed until 2030 to achieve the targets of the Paris Agreement. However, current solutions are uncertain, lack transparency, and are prone to malicious actors.

Carbon markets are trading systems in which carbon credits are sold and bought to compensate for greenhouse gas emissions. It is expected that, in the short to medium term, carbon markets will become more highly regulated, in order to introduce greater consistency, reinforce the integrity of sustainability disclosures, and respond to stakeholders' expectations that sustainability information should be transparent and comparable. Examples of these regulatory processes are outputs from the Taskforce for Nature-Based Disclosures and Taskforce for Climate-Based Disclosures.

Currently, owners or stakeholders of a nature-based asset must register with a verification body and create or register a new project describing carbon credit. Project documentation is uploaded describing details such as project name, project size, location, and average annual volume of voluntary carbon unit (1 unit=1 ton CO₂). A full review is conducted of project documentation with, typically, two to three rounds of findings and responses. Once approved by a registration body, the owner or stakeholder can sell voluntary carbon units.

“Validation” and “verification” are important steps for a nature-based project. During “validation”, a certifying body determines whether a project meets all rules and requirements of the program. Once validation has been concluded, the project proponent may submit the project for registration with the respective program. During “verification”, a certifying body confirms that the outcomes set out in the project documentation have been achieved and quantified according to the requirements of the respective standard.

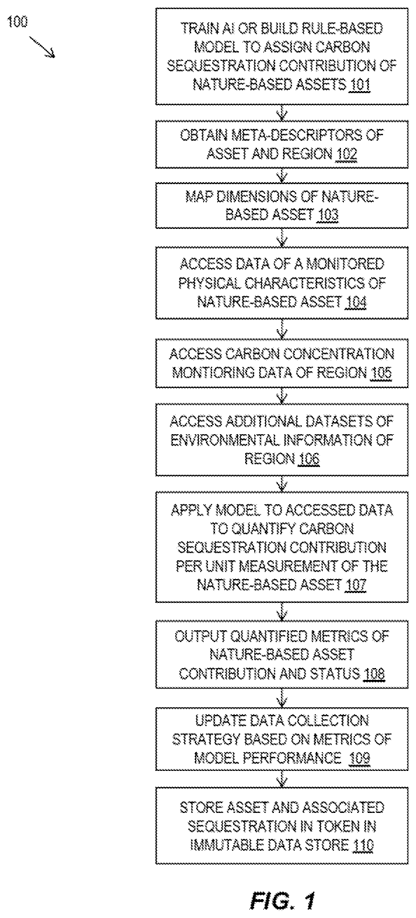

With reference to FIG. 1, a flow diagram 100 shows an example embodiment of the described method for evaluating carbon sequestration contributions of nature-based assets.

The method may include training 101 an artificial intelligence or building a rule-based model to assign carbon sequestration contribution per unit measurement of a nature-based asset in a region of a water environment. The training may include feature extraction combining: learning pertinent environmental features influencing carbon sequestration potential; estimating carbon concentration values from hydro and atmospheric environmental variables; estimating the contribution of different features to model prediction and their spatial and temporal characteristics; and extracting information on the physical and material characteristics of the asset.

The method may obtain 102 meta-descriptors of the nature-based asset. This may include details of the asset, the region being assessed, specific properties of the asset introduced (e.g., carbon absorbing geopolymers), and information on the regulatory framework or standard used for reporting carbon sequestration contribution.

The method may include mapping 103 dimensions of the nature-based asset. This may include the spatial dimensions of the asset, specific properties of the region (e.g., substrate condition), and additional natural or anthropogenic characteristics (e.g., presence or introduction of seagrass farm).

The method may include accessing 104 data of monitored physical characteristics of THE nature-based asset in the water environment. Monitoring physical characteristics of the nature-based asset in the region may include using acoustic and/or vision devices.

The method may include accessing 105 data of monitored carbon concentration in a region of a water environment. This may leverage a sensing platform and artificial intelligence-backed sensing devices. The method may also include accessing 106 additional datasets of environmental information of the water environment including monitored physical, chemical, and biological properties. Monitoring data of carbon concentration in the region of a water environment may include in-situ data and exogenous environmental data. Monitoring carbon concentration in the region of a water environment may use rapid and autonomous monitoring of water variables using in-situ data and remote sensing.

The data collection strategy may be updated based on the outputs of the model to improve the ability of the system to collect most relevant data for the model performance and carbon estimation.

The method applies 107 the trained artificial intelligence (AI) or rule-based model to assign carbon sequestration contribution per unit measurement of the nature-based asset. Applying the model may include inputting the meta-descriptors of the nature-based asset and region. Additional attributes may be specified such as the category or categories of nature-based solution being claimed (e.g., mangrove forest, seagrass farm, coral reef, oyster habitat, etc.), properties of the interventions such as material composition, and suitable regulatory frameworks and standards. The model may also ingest data from IoT sensors, water quality sampling, numerical model analysis, remote sensing estimates, drone imagery, satellite imagery, and acoustic data.

The method may output 108 metrics of nature-based asset contribution and status. The method may include applying the model to processing and classification of health indices of the nature-based asset. The method may include applying the trained artificial intelligence model to predict carbon sequestration of the asset over time.

The method may include updating 109 the data collection strategy based on modeling location sensing requirements to model locations for deployment of sensor devices in the region including increased sampling at regions of larger data variance based on statistical metrics of monitored data. This may leverage an AI component to dynamically optimize sensor placement based on statistical metrics of the data or model training. The location modeling may be applied to an autonomous surface or underwater vessel or device to deploy the sensor devices at different locations. This may include an AI-guided sampling module that guides a vessel trajectory to optimize sampling strategy (e.g. sample more heavily in regions with larger variance or regions that contribute more heavily to model prediction).

The updating 109 of the data collection strategy may monitor the contribution of different features to prediction and prioritize collection of certain features based on feature importance or contribution to prediction.

The method may store 110 a tokenized contribution of the nature-based asset in an immutable data store. The token may define the asset and associated time-varying carbon sequestration. Additional information stored in the immutable data store may include the data used to compute carbon sequestration contribution, and the framework used to estimate.

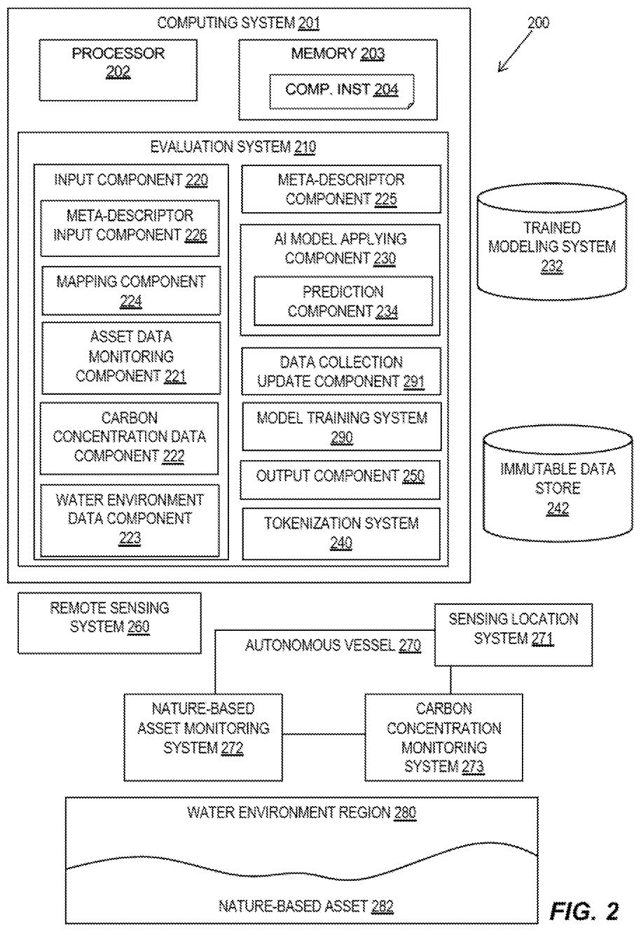

Referring to FIG. 2, a block diagram shows a system 200 as an example embodiment including the described evaluation system 210 for carbon sequestration contribution evaluation of nature-based assets located in water. In this example embodiment, a trained AI modeling system 232 is trained and applied. In other embodiments, a rule-based modeling system may be built and used.

The evaluation system 210 may be implemented on a computing system 201 that may include at least one processor 202, a hardware module, or a circuit for executing the functions of the described components which may be software units executing on the at least one processor. Multiple processors running parallel processing threads may be provided enabling parallel processing of some or all of the functions of the components. Memory 203 may be configured to provide computer instructions 204 to the at least one processor 202 to carry out the functionality of the components. The illustrated systems and components may be provided on multiple computing systems.

The evaluation system 210 includes an input component 220 for accessing monitored data, an AI model applying component 230 using a trained modeling system 232, and an output component 250 for outputting the results of the modeling.

The evaluation system 210 may have an associated a model training system 290 for training the trained modeling system 232 associated with the evaluation system 210. The model training system 290 may include training an artificial intelligence model to assign carbon sequestration contribution per unit measurement of the nature-based asset using feature extraction combining: carbon concentration estimates and drivers from data of water and/or atmospheric environments; and asset essential variables from data from asset monitoring. The training may also include characteristics of the asset and regulatory framework and standard provided as input.

The evaluation system 210 may include a meta-descriptor component 225 for providing parameters for the asset and region. The input component 220 may include a meta-descriptor input component 226 inputting meta-descriptors of the asset to the trained artificial intelligence model. The input component 220 may include a mapping component 224 for mapping dimensions of the nature-based asset.

The input component 220 may include an asset data monitoring component 221 for accessing data of monitored physical characteristics of a nature-based asset in the water environment based on the meta-descriptors. The input component 220 may include a carbon concentration data component 222 for accessing data of monitored carbon concentration in a region of a water environment based on the meta-descriptors. The input component 220 may include a water environment data component 223 for accessing data on monitored physical, chemical, and biological properties of the water environment based on the meta-descriptors of the region.

The evaluation system 210 may include a data collection update component 291 to update data collection strategy based on the performance of the model training.

The evaluation system 210 may also include a tokenization system 240 for storing tokens relating to the modeling and evaluation in an immutable data store 242. The immutable data store 242 may store nature-based asset and associated time-varying carbon sequestration or other nature-based contribution in tokens.

The evaluation system 210 may be used to evaluate a nature-based asset 282 in a water environment region 280, such as an ocean region or fresh-water region. The trained modeling system 232 may be trained and applied to inputs to assign carbon sequestration contribution or other nature-based contribution per unit (e.g. per square meter) of nature-based asset. The AI model applying component 230 may include a prediction component 234 for applying the trained modeling system 232 to predict carbon sequestration of the asset over time.

An autonomous vessel 270 may be used to obtain data relating to the nature-based asset 282 and the water environment region 280 and may be in communication with the evaluation system 210. A carbon concentration monitoring system 273 and a nature-based asset monitoring system 272 may be provided for gathering data in the regions. The autonomous vessel 270 may be an autonomous surface or underwater vessel or device that can deploy sensor devices from different locations. The autonomous vessel 270 may include an edge compute component to deploy sensors and collect and transmit data.

The autonomous vessel 270 may also include a sensing location system 271. The sensing location system 271 may include an AI-guided sampling module that guides the vessel trajectory to optimize sampling strategy (e.g. sample more heavily in regions with larger variance). The sensing location system 271 may include a sensing location optimization system to quantify the contribution of different features or subset of features to model prediction and guide sampling location and frequency based on that information. This may include capabilities to increase sampling at regions of larger data variance based on statistical metrics of monitored data or regions that make more substantial contributions to AI model training.

The carbon concentration monitoring system 273 may be for monitoring carbon concentration in the region of a water environment using rapid and autonomous monitoring of water variables using in-situ data and remote sensing. The carbon concentration monitoring system 273 may include a sensing platform and/or AI-backed sensing devices that can rapidly and autonomously measure carbon concentration and, optionally, other parameters in water. The carbon concentration monitoring system 273 may be deployed from a stationary device, e.g. a buoy with vertical samplers attached that measure chemo-physical characteristics over time. The carbon concentration monitoring system may sample other water and atmospheric variables and use an AI or rule-based model to estimate carbon concentration values from these variables.

The nature-based asset monitoring system 272 may include an acoustic and/or computer vision device that can be used to monitor physical characteristics of the nature-based asset (e.g., coral reef, mangrove reef, seagrass forest).

The system may also include a remote sensing system 260 module for remote sensing pertinent ocean color variables (e.g., temperature, salinity, Chlorophyll-a). The remote sensing system 260 may be a satellite sensing system for geospatial measurements of ocean or other water color variables providing a spatial estimate of surface measurements at daily, weekly, or monthly frequency.

The trained modeling system 232 carbon sequestration contribution or other nature-based contribution per square meter of nature-based asset based on the measures sampled by the described systems. The trained modelling system 232 provides AI-based processing of nature-based health indices, carbon sequestration contribution, and trajectory or evolution of the nature-based asset.

### Modeling System

The inputs when applying the modeling system may include at least some of the following data:
parameters of a region to be monitored;
meta-descriptors of the nature-based asset (type, age, size, status);
optional additional features (ocean model data, weather data); and
details on the regulatory framework or standards pertinent to the carbon markets or trading system where the nature-based contribution will be offered.

Meta-descriptors of the nature based asset may include location, proposed extents, type of asset (for example, coral reef, mangrove reef, seagrass, etc.), age or maturity level, status or any other descriptors. These may serve as static attributes that can inform training of AI models.

Monitored data relating to carbon concentration may be accessed by the modeling system in the modeled region may be point measurements of carbon dioxide saturation (pCO₂) made at different depths in the ocean using chemical sensors. The chemical sensors may include AI-assisted chemical sensing with software-defined sensors, such as Hyper Taste (Trademark of International Business Machines Corporation).

Monitored data relating to the nature-based asset may be accessed by the modeling system in the modeled region with measurements of primary productivity of the asset using for example Chlorophyll-a sensors, or “AI Microscope”, a small, autonomous microscope that can be placed in bodies of water to monitor plankton in situ, identifying different species and tracking their movement in three dimensions.

These monitored datasets may be extended with other commonly used ocean or hydrological datasets such as conductivity, temperature, dissolved oxygen, pH, and turbidity. Further additional geospatial datasets such as remote sensing, weather information, and public ocean model data may be used.

Using sonar or equivalent hydroacoustic capabilities, wave-length conversion may be used to map out the precise dimensions of the asset allowing for continuous quantification of reef maturity measurements and status of vegetation, fauna etc. Alternatively, this may leverage manual sampling or measurement of asset dimensions and characteristics.

The trained machine learning modeling system may be provided to assign nature-based contribution per unit measurement. This may include carbon sequestration or other environmental or ecosystem contributions. It may leverage pre-trained foundation models leveraging techniques such as the transformer architecture and pre-training across a diverse set of data.

The pre-trained AI model processes data from sensors on environmental metrics, reef physical metrics, and any other datasets to generate metrics on: carbon sequestration, biodiversity, nature-based contribution of asset; physical extents and characteristics of reef, and/or reef health or status indices. This may include capabilities to update the model using online learning capabilities. It may include capabilities to provide input to the model as textual prompts. It may include multi-modal capabilities that allows the model to ingest data from diverse data types such as time series, textual, image, graph, and point cloud.

The outputs may be tokenized within an immutable data store (e.g. Blockchain) as a verified asset that can be sold on the voluntary carbon market.

The modeling system may provide at least some of the following outputs:
carbon sequestration (or other nature-based contribution including, for example, biodiversity) per area per time period;
background or baseline level of carbon sequestration, water quality, or ecosystem services;
change or time series evolution of carbon sequestration, water quality, or ecosystem services;
physical extents and characteristics of the asset;
nature-based asset health index or status; and
changes or modifications to asset characteristics and performance.

The outputs may include quantified metrics of nature-based contribution per unit per time (e.g. per sqm per day). The quantified metrics may include: volume of carbon absorbed, contributions to improved biodiversity or ecosystem health, or improved coastal resilience or other infrastructural contribution.

The outputs may include metrics quantifying health index or status of nature based asset that may include trajectory or evolution of asset performance or early identification of potential deterioration in health (and possibly subsequent reduction in nature-based contribution). This may use specific indices of water quality such as the Index of Coastal Eutrophication (ICEP) or the National Sanitation Foundation Water Quality Index (NSF-WQI). Alternatively, appropriated proxy indicators may be used.

The outputs may be tokenized within an immutable data store (e.g. Blockchain) as a verified asset that can be sold on the voluntary carbon market.

This may be used for tokenization of nature-based contribution within a blockchain network. Some of the information tokenized may include: details on the nature-based asset and its meta descriptors; details on baseline levels of carbon sequestration or other nature-based contribution; details on changes to levels of carbon sequestration or other nature-based contribution; data on the data used to quantify those and the standards or indices adopted.

### Environment Monitoring and Sensing

Monitored data relating to carbon concentration may be obtained by point measurements of carbon dioxide saturation (pCO₂) made at different depths in the ocean using chemical sensors. The chemical sensors may include AI-assisted chemical sensing with software-defined sensors, such as HyperTaste (Trademark of International Business Machines Corporation). Potentiometric electronic tongues is used as a technology that combines miniaturization, edge computing, and AI for chemical analysis of liquids. A micro-controlled data acquisition procedure is combined with a miniaturized array of multiple electrode-posited conductive polymers. Data is fed to an AI model to identify chemical compositions.

Monitored data relating to the nature-based asset may be accessed by the modeling system in the modeled region with measurements of primary productivity of the asset using for example Chlorophyll-a sensors, or “AI Microscope”, a small, autonomous microscope that can be placed in bodies of water to monitor plankton in situ, identifying different species and tracking their movement in three dimensions. This can readily track evolving biological properties of the ocean and importantly changes to biodiversity.

Monitored data relating to the environmental status and ecosystem services of the asset may be collected by IoT or other sensing devices that measure different points of the environment. These may collect data such as conductivity, temperature, dissolved oxygen, turbidity, and pH.

Data on environmental status, carbon sequestration, and nature-based asset may be collected using autonomous or remotely operated surface or underwater vessels or devices. The trajectory of these and the associated sampling locations and time periods may be guided by an AI model based on contributions to model training or the statistical variance or descriptors of the data.

### Physical Asset Sensing

The disclosure collects information on nature-based asset characteristics, extents, health, and condition to assign nature-based contribution to a specific asset. Example monitoring can include the following techniques.

Computer vision data may be used to collect specific data on nature-based asset health and may be combined with AI microscope use to provide highly detailed measures in specific cases. This may leverage hyperspectral vision. EchoSounder technology may be used to generate high resolution maps of the water regions such as sea beds in three dimensions. These techniques may be used to continuously and autonomously (e.g. on underwater gliders) map the extents and growth of the asset such as reef.

These data can further be used to extract metrics on the asset health. For example, for an asset in the form of a reef this may be based on the principle that “a noisy reef is a healthy reef” which has been shown to possess predictive skill. This may extend research from vessel voyages to monitor underwater acoustics.

The asset sensing system may generate autonomous, continuous data characterising the spatial extent, growth, health, and biological processes of the asset.

The monitoring may leverage autonomous vessels and gliders to extend the spatial coverage to any size nature-based asset.

Remote sensing may be used including satellite or aerial measurement of the ocean surface to measure surface variables (temperature, salinity, Chlorophyll-a) at large scale. Quantification of oceanic carbon dioxide sequestration may be used to improve quantification approaches.

Data from other sources may also be used such as weather data, ocean model data, etc.

### AI Model Training and Feature Extraction

Data from environmental sensing and asset sensing are used to train an AI model to classify nature-based contribution (e.g. carbon sequestration, biodiversity) of the asset. The AI model processes point measurements of environmental metrics (CO₂, dissolved oxygen, temperature, Chl-a, pH, etc.) and geospatial (particular location) metrics (sea surface temperature, salinity, Chl-a, etc.), and creates a quantification of nature-based contribution per sqm of ocean bed.

Inputs to the model may include multi-modal datasets such as time series, geospatial or gridded data, images, text, or point cloud data. These may be ingested using pretrained foundation models, encoder-decoder architectures, autoencoder, graph neural network, or transformer-based architectures to adapt to dataset complexity and adopt the appropriate set of features for model training. Auto-AI approaches may also be used for feature extraction that processes the data using grid search approaches to extract the most relevant features and identify any possible transformations of those features to improve model prediction.

Data from environmental sensing and reef sensing are used to also train an AI model to classify the health of the asset. Features to the model may include vision data, acoustic data, text data, and other physical descriptors of the nature-based asset and a corresponding health index.

The AI model may be trained in a supervised manner with appropriate label data or in unsupervised manner using approaches such as masked self-attention.

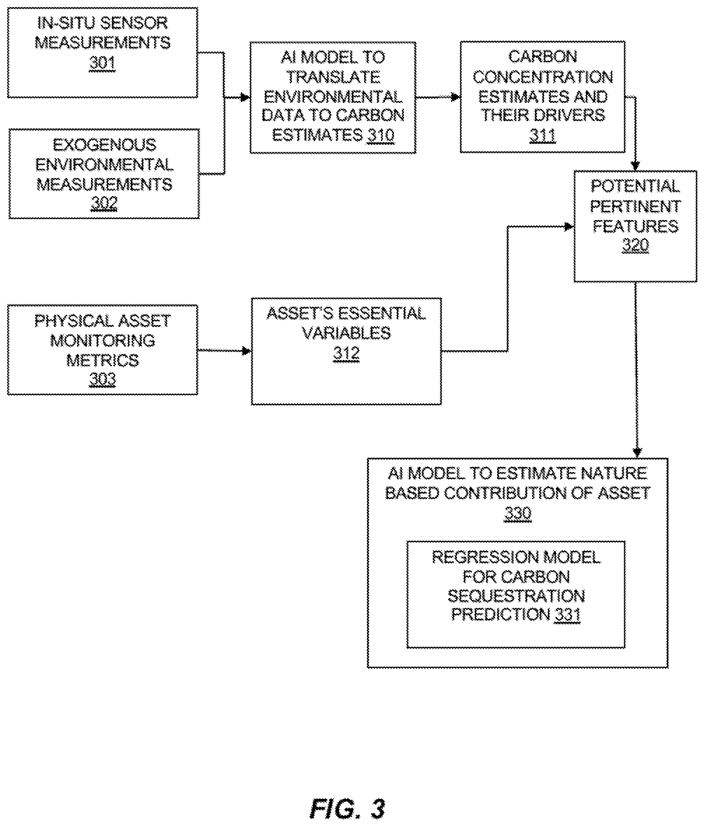

FIG. 3 shows a schematic diagram 300 showing an example embodiment of feature extraction for the AI modeling system.

In-situ sensor measurements 301 are accessed which may include: temperature, pH, DO, Salinity, Chl-a, pCO₂.

Exogenous environmental measurements 302 are accessed which may include:
1. Remote sensed SST, Salinity, Chl-a; and
2. Numerical ocean or atmospheric product data; and
3. Scientific articles or reports on the environmental status in unstructured form.

Physical asset monitoring metrics 303 are accessed which may include:
1. Visual or camera data;
2. Hydroacoustic sampling such as an echosounder;
3. High detail imagery such as hyperspectral; and
4. Natural language descriptors of the asset

The in-situ sensor measurements 301 and the exogenous environmental measurements 302 may be combined to be translated 310 by the AI model into carbon estimates to extract carbon concentration estimates 311 and their pertinent drivers.

For example, the carbon concentration estimates 311 and their pertinent drivers may include: pCO₂ estimates and drivers such as temperature, dissolved oxygen, pH, etc.

The physical asset monitoring metrics 303 may be used to extract asset essential variables 312. These may include the spatial extents and dimensions, specific nature remediation assets such as seagrass farms, kelp, or coral reefs, and specific carbon sequestering geopolymers deployed as part of the asset

For example, in the case of the physical asset being a coral reef, the asset essential variables 312 may include:
1. Hard coral cover and composition;
2. Macro-algal canopy cover; and
3. Fish diversity and abundance.

Potential pertinent features 320 may be extracted from the carbon concentration estimates 311 and their drivers and the asset's essential variables 312. This may include a manual feature extraction step to extract pertinent features, or all data may be fed to the model and pertinent features learned using approaches such as self-attention.

The potential pertinent features 320 may include:
Timeseries data of: CO2 concentration, environmental variables, asset health indices.
Meta information of: location of the asset including latitude, longitude, and depth; time; asset extents; physical properties of the asset such as vegetation cover; specific remediation activities introduced such as kelp growth or oyster beds; and any other nature-based contribution such as carbon sequestering geopolymers or enhanced remediation efforts.

The AI modeling system 330 estimates the nature based contribution of the asset. For example, for an asset in the form of a coral reef, this may include carbon sequestration based on coral reef status. It may also include the biodiversity contribution of the reef as outlined in the Kunming-Montreal Global Biodiversity Framework.

An embodiment may include a regression model 331 to predict carbon sequestration given the asset status and associated environmental status.

The described method and system leverage AI and edge-based data collection to generated reliable estimates of the nature-based contribution from different water environment assets. This leverages AI to extract the portion that are assignable to the specific assets rather than baseline environmental processes. The method and system are adaptable to different spatial and temporal scales. This provides a transparent and secure framework for verification, tokenization and sale of nature based contribution (e.g. voluntary carbon unit).

The method and system consider the number of sensor measurements that are required to train a model over an extended area of the ocean. This includes an AI system that evaluates model estimates and updates the spatial and temporal frequency of sensor measurements to optimize model performance.

Measuring, estimating, and resolving processes in the ocean is highly sensitive to spatial and temporal variation. Without adequate spatial coverage, model performance will be limited. Incorporating an AI-backed system to optimise sensor placement dynamically improved model performance. This may include monitoring the statistical properties of the collected data and prioritizing areas with higher variance or monitoring the training or fine-tuning of the AI model and prioritizing regions that contribute more heavily to model training.

The tokenization of metrics on the characteristics or descriptors of the asset or product, together with measured and modelled data on carbon sequestration contribution within an immutable data store serves to enhance the veracity and trust of the carbon credit contributions.

Using direct measurements and AI (instead of proxies), this disclosure provides a robust, defensible, quantification metric that meets current and future regulatory requirements.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

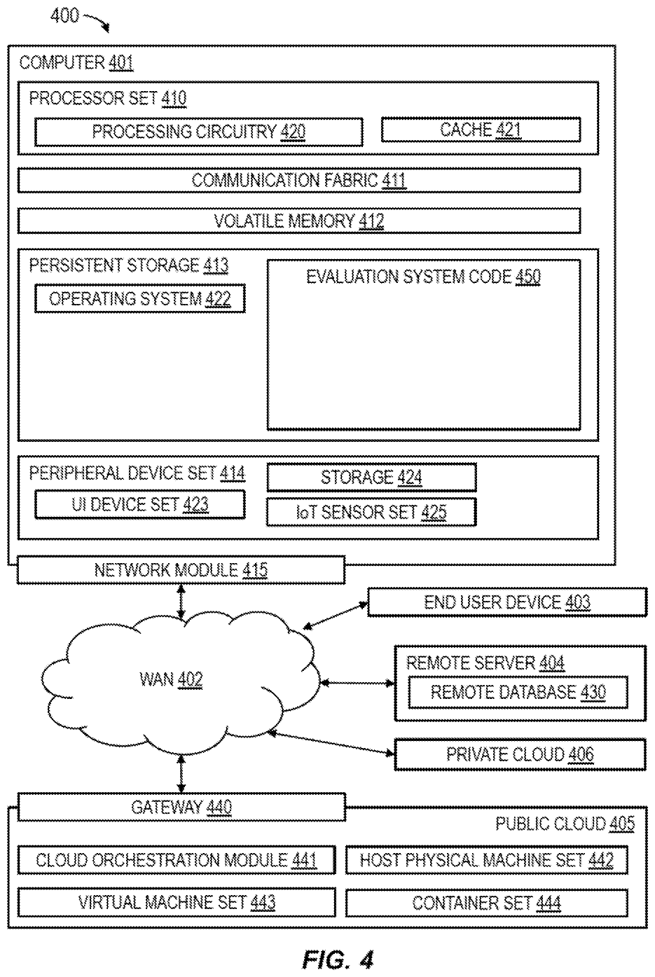

Referring to FIG. 4, computing environment 400 contains an example of an environment for the execution of at least some of the computer code involved in performing the inventive methods, such as evaluation system code in block 450. In addition to block 450, computing environment 400 includes, for example, computer 401, wide area network (WAN) 402, end user device (EUD) 403, remote server 404, public cloud 405, and private cloud 406. In this embodiment, computer 401 includes processor set 410 (including processing circuitry 420 and cache 421), communication fabric 411, volatile memory 412, persistent storage 413 (including operating system 422 and block 450, as identified above), peripheral device set 414 (including user interface (UI) device set 423, storage 424, and Internet of Things (IoT) sensor set 425), and network module 415. Remote server 404 includes remote database 430. Public cloud 405 includes gateway 440, cloud orchestration module 441, host physical machine set 442, virtual machine set 443, and container set 444.

COMPUTER 401 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 430. As is well understood in the art of computer technology, and depending upon the technology, performance of a computer-implemented method may be distributed among multiple computers and/or between multiple locations. On the other hand, in this presentation of computing environment 400, detailed discussion is focused on a single computer, specifically computer 401, to keep the presentation as simple as possible. Computer 401 may be located in a cloud, even though it is not shown in a cloud in FIG. 4. On the other hand, computer 401 is not required to be in a cloud except to any extent as may be affirmatively indicated.

PROCESSOR SET 410 includes one, or more, computer processors of any type now known or to be developed in the future. Processing circuitry 420 may be distributed over multiple packages, for example, multiple, coordinated integrated circuit chips. Processing circuitry 420 may implement multiple processor threads and/or multiple processor cores. Cache 421 is memory that is located in the processor chip package(s) and is typically used for data or code that should be available for rapid access by the threads or cores running on processor set 410. Cache memories are typically organized into multiple levels depending upon relative proximity to the processing circuitry. Alternatively, some, or all, of the cache for the processor set may be located “off chip.” In some computing environments, processor set 410 may be designed for working with qubits and performing quantum computing.

Computer readable program instructions are typically loaded onto computer 401 to cause a series of operational steps to be performed by processor set 410 of computer 401 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 421 and the other storage media discussed below. The program instructions, and associated data, are accessed by processor set 410 to control and direct performance of the inventive methods. In computing environment 400, at least some of the instructions for performing the inventive methods may be stored in block 450 in persistent storage 413.

COMMUNICATION FABRIC 411 is the signal conduction path that allows the various components of computer 401 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 412 is any type of volatile memory now known or to be developed in the future. Examples include dynamic type random access memory (RAM) or static type RAM. Typically, volatile memory 412 is characterized by random access, but this is not required unless affirmatively indicated. In computer 401, the volatile memory 412 is located in a single package and is internal to computer 401, but, alternatively or additionally, the volatile memory may be distributed over multiple packages and/or located externally with respect to computer 401.

PERSISTENT STORAGE 413 is any form of non-volatile storage for computers that is now known or to be developed in the future. The non-volatility of this storage means that the stored data is maintained regardless of whether power is being supplied to computer 401 and/or directly to persistent storage 413. Persistent storage 413 may be a read only memory (ROM), but typically at least a portion of the persistent storage allows writing of data, deletion of data and re-writing of data. Some familiar forms of persistent storage include magnetic disks and solid state storage devices. Operating system 422 may take several forms, such as various known proprietary operating systems or open source Portable Operating System Interface-type operating systems that employ a kernel. The code included in block 450 typically includes at least some of the computer code involved in performing the inventive methods.

PERIPHERAL DEVICE SET 414 includes the set of peripheral devices of computer 401. Data communication connections between the peripheral devices and the other components of computer 401 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 423 may include components such as a display screen, speaker, microphone, wearable devices (such as goggles and smart watches), keyboard, mouse, printer, touchpad, game controllers, and haptic devices. Storage 424 is external storage, such as an external hard drive, or insertable storage, such as an SD card. Storage 424 may be persistent and/or volatile. In some embodiments, storage 424 may take the form of a quantum computing storage device for storing data in the form of qubits. In embodiments where computer 401 is required to have a large amount of storage (for example, where computer 401 locally stores and manages a large database) then this storage may be provided by peripheral storage devices designed for storing very large amounts of data, such as a storage area network (SAN) that is shared by multiple, geographically distributed computers. IoT sensor set 425 is made up of sensors that can be used in Internet of Things applications. For example, one sensor may be a thermometer and another sensor may be a motion detector.

NETWORK MODULE 415 is the collection of computer software, hardware, and firmware that allows computer 401 to communicate with other computers through WAN 402. Network module 415 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 415 are performed on the same physical hardware device. In other embodiments (for example, embodiments that utilize software-defined networking (SDN)), the control functions and the forwarding functions of network module 415 are performed on physically separate devices, such that the control functions manage several different network hardware devices. Computer readable program instructions for performing the inventive methods can typically be downloaded to computer 401 from an external computer or external storage device through a network adapter card or network interface included in network module 415.

WAN 402 is any wide area network (for example, the internet) capable of communicating computer data over non-local distances by any technology for communicating computer data, now known or to be developed in the future. In some embodiments, the WAN 402 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 403 is any computer system that is used and controlled by an end user (for example, a customer of an enterprise that operates computer 401), and may take any of the forms discussed above in connection with computer 401. EUD 403 typically receives helpful and useful data from the operations of computer 401. For example, in a hypothetical case where computer 401 is designed to provide a recommendation to an end user, this recommendation would typically be communicated from network module 415 of computer 401 through WAN 402 to EUD 403. In this way, EUD 403 can display, or otherwise present, the recommendation to an end user. In some embodiments, EUD 403 may be a client device, such as thin client, heavy client, mainframe computer, desktop computer and so on.

REMOTE SERVER 404 is any computer system that serves at least some data and/or functionality to computer 401. Remote server 404 may be controlled and used by the same entity that operates computer 401. Remote server 404 represents the machine(s) that collect and store helpful and useful data for use by other computers, such as computer 401. For example, in a hypothetical case where computer 401 is designed and programmed to provide a recommendation based on historical data, then this historical data may be provided to computer 401 from remote database 430 of remote server 404.

PUBLIC CLOUD 405 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 405 is performed by the computer hardware and/or software of cloud orchestration module 441. The computing resources provided by public cloud 405 are typically implemented by virtual computing environments that run on various computers making up the computers of host physical machine set 442, which is the universe of physical computers in and/or available to public cloud 405. The virtual computing environments (VCEs) typically take the form of virtual machines from virtual machine set 443 and/or containers from container set 444. It is understood that these VCEs may be stored as images and may be transferred among and between the various physical machine hosts, either as images or after instantiation of the VCE. Cloud orchestration module 441 manages the transfer and storage of images, deploys new instantiations of VCEs and manages active instantiations of VCE deployments. Gateway 440 is the collection of computer software, hardware, and firmware that allows public cloud 405 to communicate through WAN 402.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 406 is similar to public cloud 405, except that the computing resources are only available for use by a single enterprise. While private cloud 406 is depicted as being in communication with WAN 402, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 405 and private cloud 406 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

1. A computer-implemented method for evaluating carbon sequestration contributions of nature-based assets located in water environments, the method comprising:
obtaining meta-descriptors of a nature-based asset and a region of a water environment in which the asset is located;
mapping dimensions of the nature-based asset;
accessing data of monitored physical characteristics of the nature-based asset in the water environment based on the meta-descriptors of the asset;
accessing data of monitored carbon concentration in the region of the water environment based on the meta-descriptors of the region;
accessing data on monitored physical, chemical, and biological properties of the water environment based on the meta-descriptors of the region; and
applying a model to the accessed data to assign a carbon sequestration contribution per unit measurement of the nature-based asset.

2. The method of claim 1, comprising:
storing a tokenized contribution of the nature-based asset in an immutable data store.

3. The method of claim 1, wherein accessing data of monitored carbon concentration in a region of a water environment includes in-situ data and exogenous environmental data.

4. The method of claim 1, comprising:
monitoring carbon concentration in the region of a water environment using rapid and autonomous monitoring of water variables using in-situ data and remote sensing; and
monitoring physical characteristics of the nature-based asset in the region using acoustic and vision devices.

5. The method of claim 1, comprising applying the model by inputting parameters of the meta-descriptors of the asset and region.

6. The method of claim 1, comprising:
location modeling to model locations for deployment of sensor devices in the region including increased sampling at regions.

7. The method of claim 1, comprising applying the model to processing and classification of health indices of the nature-based asset and/or to predict carbon sequestration of the asset over time.

8. The method of claim 1, comprising updating data collection requirements based on metrics of a model's performance.

9. The method of claim 1, comprising:
training the model in a form of an artificial intelligence model to assign carbon sequestration contribution per unit measurement of the nature-based asset using feature extraction combining:
carbon concentration estimates and drivers from data of water environments; and
asset essential variables from data from asset monitoring.

10. The method of claim 9, wherein the training includes the feature extraction combining:
learning pertinent environmental features influencing carbon sequestration potential;
estimating carbon concentration values from hydro and atmospheric environmental variables;
estimating the contribution of different features to model prediction and their spatial and temporal characteristics; and
extracting information on the physical and a material characteristics of the asset.

11. The method of claim 9, wherein feature extraction includes point and geospatial measurements of environmental metrics and labels corresponding to nature-based contribution on a high resolution grid.

12. A system for evaluating carbon sequestration contributions of nature-based assets located in water environments, comprising:
a processor and a memory configured to provide computer program instructions to the processor to execute the function of one or more components:
a meta-descriptor component for obtaining meta-descriptors of a nature-based asset and a region of a water environment in which the asset is located;
a mapping component for mapping dimensions of the nature-based asset;
an asset data monitoring component for accessing data of monitored physical characteristics of the nature-based asset in the water environment based on the meta-descriptors;
a carbon concentration data component for accessing data of monitored carbon concentration in the region of the water environment based on the meta-descriptors;
a water environment data component for accessing data on monitored physical, chemical, and biological properties of the water environment based on the meta-descriptors of the region; and
a model applying component for applying a model to assign carbon sequestration contribution per unit measurement of the nature-based asset.

13. The system of claim 12, comprising:
a tokenization component for storing a tokenized contribution of the nature-based asset in an immutable data store.

14. The system of claim 12, comprising a carbon concentration monitoring system for monitoring carbon concentration in the region of a water environment using rapid and autonomous monitoring of water variables using in-situ data and remote sensing.

15. The system of claim 12, comprising an asset monitoring system for monitoring physical characteristics of the nature-based asset in the region using acoustic and/or vision devices.

16. The system of claim 12, comprising a data collection update component to update data collection strategy based on a performance of the model's training.

17. The system of claim 12, comprising a sensing location modeling system for modeling locations for deployment of sensor devices in the region including increased sampling at regions that contribute more heavily to training or fine-tuning of the model.

18. The system of claim 17, comprising communication with an autonomous surface or underwater vessel or device to deploy the sensor devices at different locations in the region.

19. The system of claim 12, comprising a model training system for:
training the model in a form of an artificial intelligence model to assign carbon sequestration contribution per unit measurement of the nature-based asset using feature extraction combining:
carbon concentration estimates and drivers from data of water environments; and
asset essential variables from data from asset monitoring.

20. A computer program stored on a computer readable medium and loadable into an internal memory of a digital computer, comprising software code portions, when said program is run on a computer, for performing a method comprising:
obtaining meta-descriptors of a nature-based asset and a region of a water environment in which the asset is located;
mapping dimensions of the nature-based asset;
accessing data of monitored physical characteristics of the nature-based asset in the water environment based on the meta-descriptors of the asset;
accessing data of monitored carbon concentration in the region of the water environment based on the meta-descriptors of the region;
accessing data on monitored physical, chemical, and biological properties of the water environment based on the meta-descriptors of the region; and
applying a model to the accessed data to assign a carbon sequestration contribution per unit measurement of the nature-based asset.
